(19) United States
(12) Patent Application Publication
(10) Pub. No.: US 2006/0127896 A1
(43) Pub. Date: Jun. 15, 2006

(54) Materials and methods for treating cancer

(76) Inventors: Aylwin Ng, Singapore (SG); Jing P. Tang, Singapore (SG); Kam Man Hui, Singapore (SG); Christopher H. K. Goh, Singapore (SG)

Correspondence Address: JENNIFER M MCCALLUM, PH D, ESQ;THE MCCALLUM LAW FIRM, LLC, 685 BRIGGS STREET, PO BOX 929, ERIE, CO, 80516, US

(21) Appl. No.: 10/502,470

(22) PCT Filed: Jan. 23, 2003
(86) PCT No.: PCT/GB03/00329

§ 371 Date: Dec. 3, 2004

(30) Foreign Application Priority Data
Jan. 23, 2002 (GB) 02014983

(51) Int. Cl. C12Q 1/68; G01N 33/574

(52) U.S. Cl. 435/6; 435/7.23

(57) ABSTRACT

The invention is concerned with the detection and treatment of nasopharyngeal carcinoma (NPC) based on differential gene expression in these cells. Specifically, the invention provides details of differentially expressed genes in NPC which serve to detect the presence or risk of the disease and its clinical type. The invention also provides methods of treating NPC in association with chemo or radiotherapy.

The present invention relates to materials and methods for treating cancer based on the differential gene expression in cancer cells. Particularly, but not exclusively, the present invention provides materials and methods for diagnosing and treating nasopharyngeal carcinoma.

Human nasopharyngeal carcinoma (NPC) arises in the surface epithelium of the posterior nasopharynx and associated with a high frequency of neck and distant metastases. NPC has a high incidence in certain regions of Southeast China, Southeast Asia, Taiwan, East Africa, and Alaska (Marks et al., 1998). The peak incidence for NPC occurs at the fourth to fifth decade of life. In Singapore, NPC is the fifth most prevalent cancer amongst males of Chinese descent having an annual incidence rate of 14.3 per 100,000 (Chia et al., 2000). Clinically, NPC is most commonly treated by ionizing radiation (Lee et al., 1992; Marks et al., 1998).

The World Health Organization (WHO) has classified NPC into three categories according to the degree of differentiation (Marks et al., 1998). Type I refers to squamous cell carcinomas which are highly differentiated with characteristic epithelial growth patterns and intra- and extra-cellular keratin filaments. Non-keratinizing WHO type II carcinomas retain epithelial cell shape and growth patterns. WHO type III undifferentiated carcinomas, on the other hand, produce no keratin and have no distinctive growth pattern. WHO-I keratinizing squamous cell carcinoma comprised 75% of the U.S. nasopharyngeal carcinoma cases and were found most in U.S.-born, non-Hispanic whites. WHO-II non-keratinizing and WHO-III undifferentiated carcinomas of the nasopharynx comprised the remaining 25% of NPC and were more common in Asians, Clinically, Asians were reported to have the highest proportion of radioresponsive WHO-II nonkeratinizing and WHO-III undifferentiated carcinomas of the nasopharynx and better survival in comparison to African-Americans and Hispanic and non-Hispanic whites, who had the greatest number of the less radioresponsive kertinizing squamous cell carcinomas of the nasopharynx. The 5-year relative survival was reported to be 65% for the nonkeratinizing and undifferentiated carcinomas of the nasopharynx and 37% for the keratinizing variety (Marks et al., 1998).

Epstein-Barr virus (EBV) has been demonstrated to be closely associated with NPC (Mutirangura et al., 1998; Chen et al., 1998). The WHO type II and III NPC have been reported to be associated with EBV infection. In WHO type II and III NPC patients, they have elevated IgG and IgA levels to the EBV viral capsid antigen (VCA) as well as the diffuse component of the early antigen (Zong et al., 1992; Sigel et al., 1994). In contrast, patients with the WHO type I well-differentiated carcinomas have similar EBV serologic profiles as that of the control populations and did not appear to have a special association with EBV infection. Furthermore, molecular studies showed that EBV genomes were clearly demonstrable in the malignant epithelial tumour cells of all three WHO types of NPC. Northern blot analysis also demonstrated the expression of EBV gene products involved in the latent and lytic cycles in biopsies obtained from NPC patients (Busson et al., 1992). Nevertheless, direct evidence to show that EBV being the etiological agent for NPC has been difficult and has yet to be established.

Another important feature of NPC is that the NPC tumours are characterized histopathologically by a heavy infiltration of non-malignant lymphocytes. A significant proportion of these tumour-infiltrated lymphocytes (TILs) have been shown to be T cells (Huang et al., 1999). The production of certain cytokines by these TILs might contribute to tumour growth during the development of NPC (Huang et al., 1999, Tang et al., 2001).

NPC carcinogenesis possibly reflects the accumulation of multiple genetic, dietary, and viral-related events that alters the normal functions of oncogenes and tumour suppressor genes (Gray and Collins, 2000; Williams, 2000). Extensive molecular analyses including karyotyping and comparative genomic hybridization (CGH) studies (Chien et al., 2001; Fang et al., 2001) have suggested that NPC arises as a multistep process. Genome-wide studies by allelotyping and CGH have detected high frequencies of genetic abnormalities on chromosomes 3p, 9p, 11q, 12q, 13q, and 14q in NPC. This data suggested the presence of a potential NPC-related tumour suppressor gene(s) to a region at 3p21.3 where the RASSFlA gene is located (Lo et al., 2001). The correlation of promoter hypermethylation with loss of RASSFlA gene expression was recently reported in NPC cells (Lo et al., 2001). Dietary exposures were found to play a role in the overall altered risk of developing specific histologic subsets of NPC. The risk of nonkeratinizing and undifferentiated tumours of the nasopharynx was increased in frequent consumers of preserved meats containing high levels of N-nitroso compounds (Farrow et al., 1998). The risk of differentiated squamous cell carcinoma, but not other histologic types, was significantly reduced in individuals with vitamin C intake (Farrow et al., 1998). This association was markedly stronger among non-smokers and former smokers than among current smokers (Farrow et al., 1998). Furthermore, titres of EBV antibody to early antigen and viral capsid antigen were found to be elevated in patients with non-keratinizing and undifferentiated carcinomas of the nasopharynx whereas the titers of these antibodies are comparable between the controls and patients with the keratinizing variety (Neel, 1985 and 1986). However, the molecular basis between the kertinizing squamous cell carcinomas of the nasopharynx and the relatively more radioresponsive nonkeratinizing and undifferentiated carcinomas of the nasopharynx have not been studied systematically.

To try to understand the molecular differences between kertinizing squamous cell carcinomas of the nasopharynx and the nonkeratinizing and undifferentiated carcinomas of the nasopharynx, the present inventors have employed cDNA microarrays to identify genes that might potentially be involved in the carcinogenesis of human NPC. The inventors have determined a small number of genes that are differentially expressed in undifferentiated and differentiated human NPC. Specifically, the inventors have found that fifteen genes were differentially up-regulated in the undifferentiated CNE-2 NPC cells, while six gene were specifically up-regulated in the well differentiated HK1 cells.

One of the genes identified to be specifically up-regulated in the undifferentiated human NPC cell line CNE-2 is the human imprinting gene H19. Interestingly, H19 is not expressed in the well-differentiated human HK1 NPC cells. Northern blot and in situ hybridization analyses also confirmed that the H19 gene is strongly expressed in the undifferentiated CNE-2 human NPC cell line but not in the well-differentiated HK1 human NPC cell line. Furthermore, the inventors have demonstrated that de-regulation of the H19 gene expression in the well-differentiated human HK1 NPC cells could be induced by the hypomethylation of CpG sites of the H19 promoter region. The inventors believe that hypermethylation of gene promoter regions may therefore be an important epigenetic event that plays a role in the differentiation of human NPC cells and the transcriptional silencing of imprinted genes.

Thus, at its most general, the present invention provides materials and methods for diagnosing and treating nasopharyngeal carcinoma (NPC). The invention further provides methods of screening for agents or therapeutic targets that may be used in the treatment or diagnosis of nasopharyngeal carcinoma.

Knowledge of the differential expression of certain genes in the different types of NPC, provides for the first time a tool for diagnosing NPC or a risk of NPC. This diagnosis may be independent of histology studies or may be used to complement histology studies.

Further and very importantly, the knowledge of differential gene expression enables diagnosis of the type of NPC, thus ensuring that the appropriate treatment is given.

In a first aspect of the present invention, there is provided a method for determining the presence or risk of a NPC in a patient comprising the steps of
(a) obtaining expression products from a nasopharyngeal cell obtained from a patient suspected of having or at risk of having a NPC;
(b) contacting said expression products with a binding members capable of binding to expression products corresponding to one or more genes identified in Table 1; and
(c) determining the presence or risk of NPC in said patient based on the binding of the expression products from said nasopharyngeal cell to the one or more binding members.

The presence or up-regulation of an expression product may be determined by comparing the presence or level of the expression product obtained from the cell under test with those from an appropriate control cell. Ideally, the control cell would be a “normal”, i.e. non-cancerous epithelial cell from the nasopharynx. These cells could also be obtained from the patient under examination. Normal epithelial cells from other parts of the body could also be used. An alternative to the analysis of a control cell is the production of expression standards that could be used as a control to compare with the expression level or pattern from the cell under test. Such standards may be produced by analysing a collection of samples to determine a “standard” expression level or pattern of one or more products in normal cells. This is discussed in more detail below.

As mentioned above, the method according to the first aspect of the invention is not only particularly suited for classifying a nasopharyngeal sample as normal or malignant, but also classifying the particular type of NPC.

Thus, in one embodiment, the invention provides a method for determining the type of NPC, e.g. differentiated or undifferentiated by detecting the differentially up-regulated expression of at least one gene identified in Table 1.

The expression product may be a transcribed nucleic acid sequence or the expressed polypeptide. The transcribed nucleic acid sequence may be RNA, mRNA or cDNA produced from mRNA.

The binding member may a complementary nucleic acid sequence which is capable of specifically binding to the transcribed nucleic acid under suitable hybridisation conditions.

Where the expression product is the expressed protein, the binding member is preferably an antibody or a molecule comprising an antibody binding domain specific for said expressed polypeptide.

The binding member may be labelled for detection purposes using standard procedures known in the art.

Preferably, the binding member is fixed to a solid support. The expression products may then be passed over the solid support, thereby bringing them into contact with the binding member. The solid support may be a glass surface, e.g. a microscope slide; beads (Lynx); or fibre-optices. In the case of beads, each binding member may be fixed to an individual bead and contacted with the expression products in solution.

The present inventors have successfully used a nucleic acid microarray comprising a plurality of nucleic acid sequences fixed to a solid support. By passing nucleic acid sequences representing expressed genes, over the microarray, they were able to create an expression profile characteristic of NPC and furthermore, the type of NPC.

Various methods exist in the art for determining expression profiles for particular gene sets and these can be applied to the present invention. For example, bead-based approaches (Lynx) or molecular bar-codes (Surromed) are known techniques. In these cases, each binding member is attached to a bead or “bar-code” that is individually readable and free-floating to ease contact with the expression products. The binding of the binding members to the expression products (targets) is achieved in solution, after which the tagged beads or bar-codes are passed through a device (e.g. a flow-cytometer) and read.

A further known method of determining expression profiles is instrumentation developed by Illumina, namely, fibre-optics. In this case, each binding member is attached to a specific “address” at the end of a fibre-optic cable. Binding of the expression product to the binding member may induce a fluorescent change, which is readable by a device at the other end of the fibre-optic cable.

The present invention further provides a nucleic acid micro-array for determining the presence or risk of NPC in an individual, comprising a solid support housing a plurality of nucleic acid sequences, said nucleic acid sequences being capable of specifically binding to expression products of one or more genes identified in Table 1. The classification of the sample will lead to the diagnosis of NPC and or the classification of the NPC in the individual.

Typically, high density nucleic acid sequences, usually cDNA or oligonucleotides, are fixed onto very small, discrete areas or spots of a solid support. The solid support is often a microscopic glass side or a membrane filter, coated with a substrate (or chips). The nucleic acid sequences are delivered (or printed), usually by a robotic system, onto the coated solid support and then immobilized or fixed to the support.

In a preferred embodiment, the expression products derived from the sample are labelled, typically using a fluorescent label, and then contacted with the immobilized nucleic acid sequences. Following hybridization, the fluorescent markers are detected using a detector, such as a high resolution laser scanner.

A binding profile indicating a pattern of gene expression (expression profile) is obtained by analysing the signal emitted from each discrete spot with digital imaging software. The pattern of gene expression of the experimental sample can then be compared with that of a control (i.e. an expression profile from a normal tissue sample) for differential analysis.

As mentioned above, the control or standard, may be one or more expression profiles previously judged to be characteristic of normal or malignant cells. These one or more expression profiles may be retrievably stored on a data carrier as part of a database. However, it is also possible to introduce a control into the assay procedure. In other words, the test sample may be “spiked” with one or more “synthetic tumour” or “synthetic normal” expression products which can act as controls to be compared with the expression levels of the genetic identifiers in the test sample.

Most microarrays utilize two fluorophores, typically, the most commonly used fluorophores are Cy3 (green channel excitation) and Cy5 (red channel excitation). The object of the micro-array image analysis is to extract hybridization signals from each expression product. Signals are measured as absolute intensities for a given target (essentially for arrays hybridized to a single sample) or as ratios of two expression products, (e.g. sample and control) with different fluorescent labels, representing two separate treatments to be compared with one probe as an internal control.

The micro-array in accordance with the present invention preferably comprises a plurality of discrete spots, each spot containing one or more oligonucleotides and each spot representing a different binding member for an expression product of a gene selected from Table 1.

In a second aspect of the present invention, there is provided a method of creating an expression profile characteristic of NPC or a particular type of NPC, said method comprising
(a) obtaining expression products from a NPC cell obtained from a patient
(b) contacting said expression products with a plurality of binding members capable of specifically binding to expression products of one or more genes identified in Table 1;
(c) determining the binding of said expression products with the binding members so as to create an expression profile characteristic of the NPC cell.

The invention further provides a nucleic acid (RNA or cDNA) expression profile database comprising expression data characteristic of a NPC or type of NPC, said data being obtained from analysis of a plurality of oligonucleotide microarrays showing nucleic acid distribution characteristic of NPC or a type of NPC, for use in diagnosis.

The present invention further provides a diagnostic tool for diagnosing a NPC or type of NPC comprising an oligonucleotide microarray, said microarray having a solid support housing a plurality of oligonucleotide sequences, said oligonucleotides individually comprising nucleic acid sequence capable of specifically binding to expressed nucleic acid of a plurality of genes identified in Table 1.

In a third aspect of the present invention, there is provided a kit for determining the presence or type of NPC in a biological sample, said kit comprising a one or more binding members capable of specifically binding to an expression product of one or more genes identified in Table 1, and a detection means. The biological sample is preferably cell extract.

Preferably, the one or more binding members (antibody binding domains or nucleic acid sequences) in the kit is fixed to a solid support. The detection means is preferably a label (radioactive or dye e.g. fluorescent dye) that detects when a binding member has bound to an expression product.

Preferably, the one or more binding members include a binding member capable of specifically binding to an expression product of H19 or CDKNIC. Both of these genes serve as convenient markers for undifferentiated human NPC. As H19 does not produce a protein product, the expression product will be mRNA. In the case of CDKNIC, the expression product can be mRNA or the resulting protein product.

As mentioned above, type II and type III undifferentiated NPC are more responsive to radiotherapy and consequently there is a better survival rate in patients suffering from these types of NPC. The present inventors have determined a number of genes that are up-regulated in undifferentiated NPC as opposed to differentiated, type I NPC (see Table 1). These genes include R19 and CDKN1C.

The inventors have further determined a number of genes that are up-regulated in type I differentiated cells as opposed to undifferentiated (type II or type III) cells (see Table 1).

Not only does the knowledge of this differential expression lead to extremely useful diagnostic methods, but it also provides new approaches in the treatment of NPC, particularly Type I which has had limited treatment success in the past.

The inventors have surprisingly found that the promoter region of the H19 gene is highly methylated in differentiated cells whereas no methylation is seen in the same region in undifferentiated cells, The inventors have further shown that demethylation of this region leads to the expression of the H19 gene in differentiated cells.

This exciting discovery provides a way to change the differential expression of genes characteristic of different types of NPC and render the cells more susceptible to treatment, e.g. radiotherapy.

Thus, in a fourth aspect of the invention there is provided a method of treating a patient with or at risk from NPC comprising administering a demethylation agent, e.g. 5′aza-2′-deoxycytidine, in association with a cancer treatment, e.g. chemo or radiotherapy.

The invention also provides the use of a demethylation agent for preparing a medicament for treating nasopharyngeal carcinoma in association with chemo or radiotherapy.

It is preferred that the demethylation agent is used in the treatment of type I NPC.

The inventors' findings that differentiated and undifferentiated forms of NPC have different gene expression, enables the development of a method for screening for substances capable of treating NPC and particularly, substances capable of selectively treating different types of NPC.

Thus, in a fifth aspect, there is provided a method of screening for substances capable of treating NPC in a patient, said method comprising
(a) over-expressing in a cell one or more genes identified in Table 1,
(b) contacting said cell with a test substance;
(c) determining the effect of said test substance on said cell as compared to the effect of said test substance on a comparable cell absent of the over-expression of said one or more genes; and
(d) identifying said test substance as a substance capable of treating NPC.

The method may further comprise the step of producing a pharmaceutical composition comprising the substance identified in step (d).

The one or more genes may be over-expressed by inserting into said cell nucleic acid capable of expressing expression products characteristic of said genes.

Depending on the screening method, it may be preferably to choose genes known to be up-regulated in either differentiated NPC or undifferentiated NPC. Further, depending on the substance under test, it may be preferable to choose those genes known in produce a protein product, e.g. CDKNIC.

In a preferred embodiment, the one or more genes being expressed include CDKN1C.

The method may also include the treatment of the cell over-expressing the one or more genes identified in Table 1 with a demethylation agent in association with the test substance.

As an alternative to recombinantly producing a cell over-expressing one or more genes characteristic of the different types of NPC, a NPC cell (Type I, II or III) could be used directly. Although this would provide valuable information concerning the effect of the test substance, further tests may be needed to identify the specific gene target.

Aspects and embodiments of the present invention will now be illustrated, by way of example, with reference to the accompanying figures. Further aspects and embodiments will be apparent to those skilled in the art. All documents mentioned in this text are incorporated herein by reference.

In the figures:

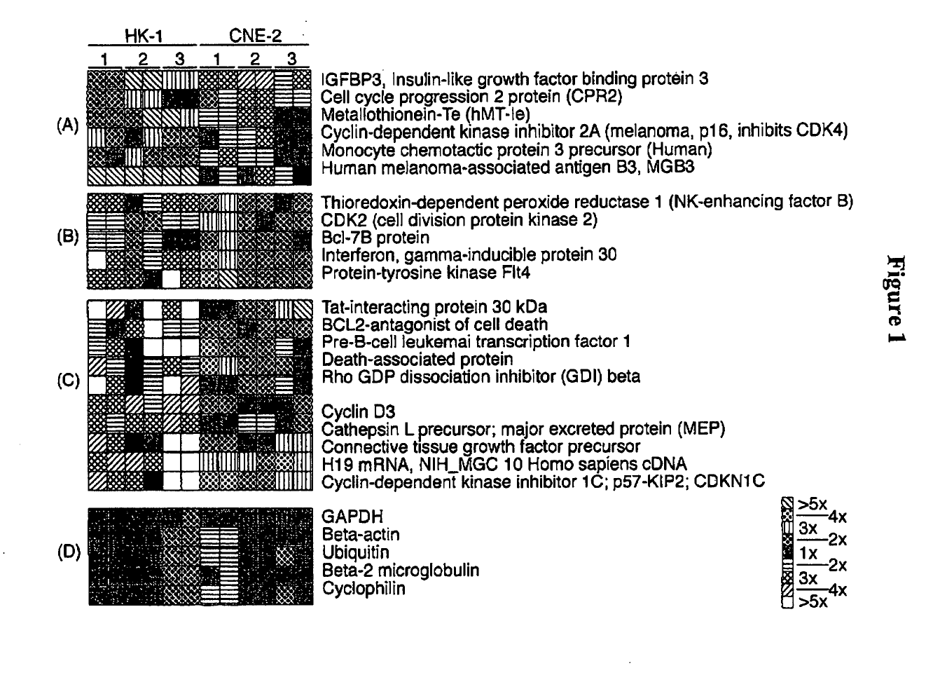

FIG. 1

Comparative microarray analyses of gene expression between undifferentiated NPC (CNE-2) cells and well-differentiated NPC (HK1) cells, from three experiments (1 to 3) performed in duplicates. cDNAs derived from CNE-2 cells or HK1 cells were labelled with Cy5 (pseudo-colored red on scanning) and reference cDNA (pooled from 10 cell lines) with Cy3 (pseudo-colored green). Log₂-transformed median-centered Cy5:Cy3 ratios are calculated using the Cluster program. These ratios are a measure of relative gene expression in each experimental sample and are displayed as a spectrum of graded colors from red through black to green, according to a color bar shown at the bottom (using the TreeView program). Unfortunately, this cannot be represented in the block and white figures accompanying this specification. Instead, line drawing has been used to try to represent the spectrum of graded colours. Red ( [symbol] )

represents a Cy5:Cy3 ratio that is higher than the median for a particular gene across experimental samples. Green ( [symbol] )

or black ( [symbol] )

represents a Cy5:Cy3 ratio that is lower than or equivalent to the median for the gene across experimental samples, respectively. Four gene clusters (A to D) are represented, showing: (A) genes that are expressed at higher levels in HK1 than in CNE-2 cells, (B and C) genes that are expressed at higher levels in CNE-2 cells than in HK1, and (D) internal ‘house-keeping’ control genes.

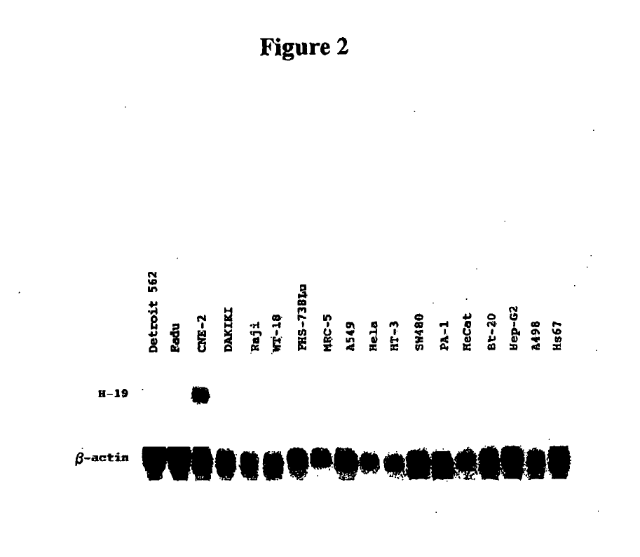

FIG. 2

Northern blot analysis of polyA⁺ RNA purified from 18 different human tumor cell lines (Detroit 562, Fadu, CNE-2, DAKIKI, Raji, WT-18, FHS-738Lu, MRC-5, A549, HeLa, HT-3, SW480, PA-1, HeCat, Bt-20, Hep-G2, A498 and Hs67). 5 μg of polyA⁺ RNA was subjected to electrophoresis in 1% formaldehyde-containing agarose gel, transferred to nylon membrane and probed for H19 and β-actin as described in ‘Materials and Methods’.

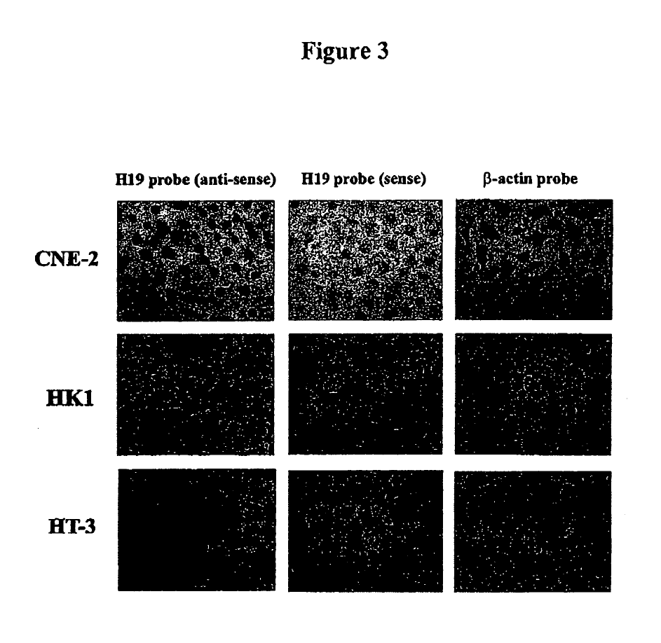

FIG. 3

In situ hybridization of cell lines derived from undifferentiated NPC (CNE-2), well-differentiated NPC (HK1) and cervical carcinoma (HT-3) using β-actin probe and H19 sense and anti-sense probes as described in ‘Materials and Methods’. Photographs were taken at magnification ×400.

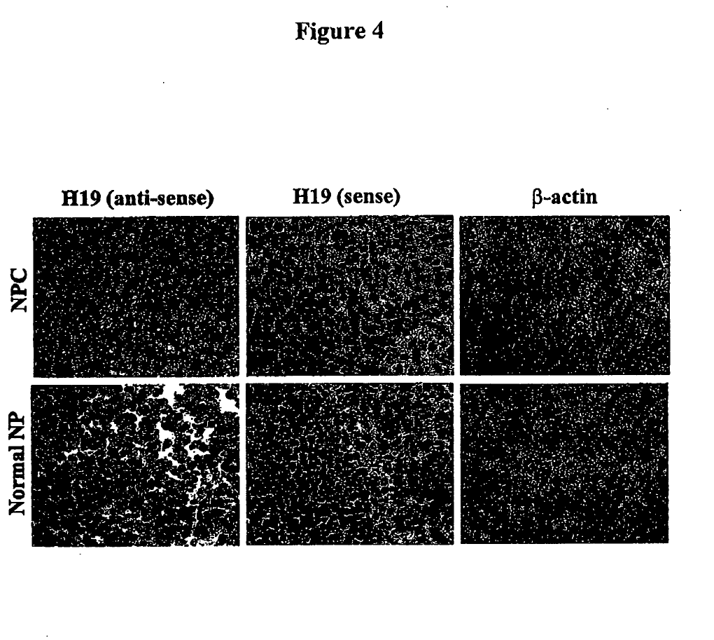

FIG. 4

In situ hybridization of primary human tissues from normal nasopharynx (NP) and undifferentiated carcinoma of the nasopharynx (NPC). DIG-labelled probes specific for H19 or β-actin were used as described in ‘Materials and Methods’. Photographs were taken at magnification ×400.

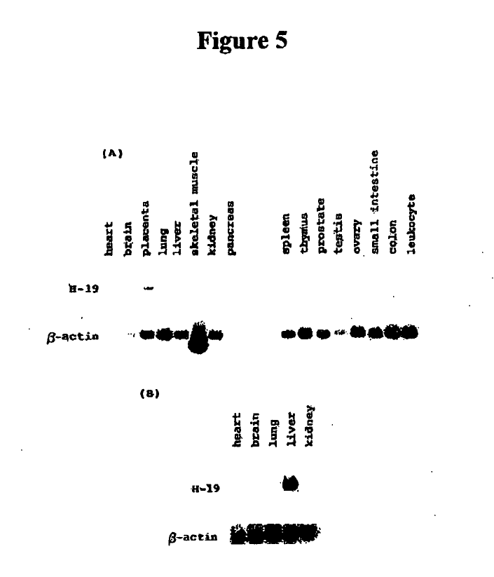

FIG. 5

Northern blot analysis of polyA⁺ RNA from (A) sixteen adult tissues (heart, brain, placenta, lung, liver, skeletal muscle, kidney, pancreas, spleen, thymus, prostate, testis, ovary, small intestine, colon and leukocyte) and (B) five fetal tissues (heart, brain, lung, liver, kidney) on nylon membrane (MTN Blots, Clontech). Blots were probed for H19 and β-actin as described in ‘Materials and Methods’.

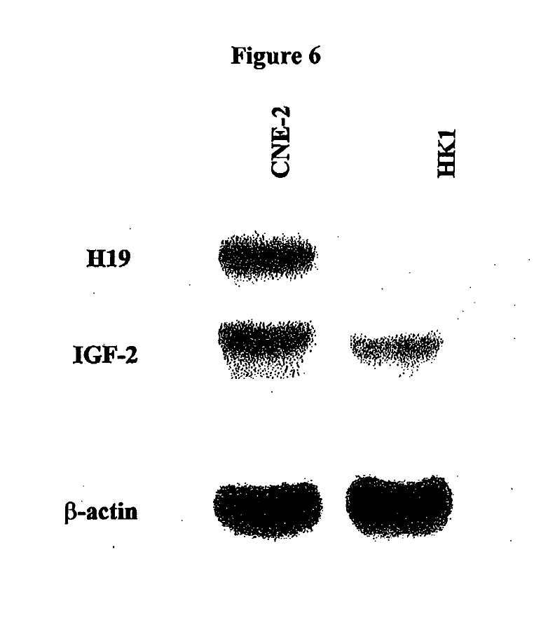

FIG. 6

Northern blot analysis of RNA from human cell lines, CNE-2 and HK1, derived from undifferentiated and well-differentiated carcinomas of the nasopharynx respectively, using probes for H19, insulin-like growth factor 2 (IGF-2) and β-actin.

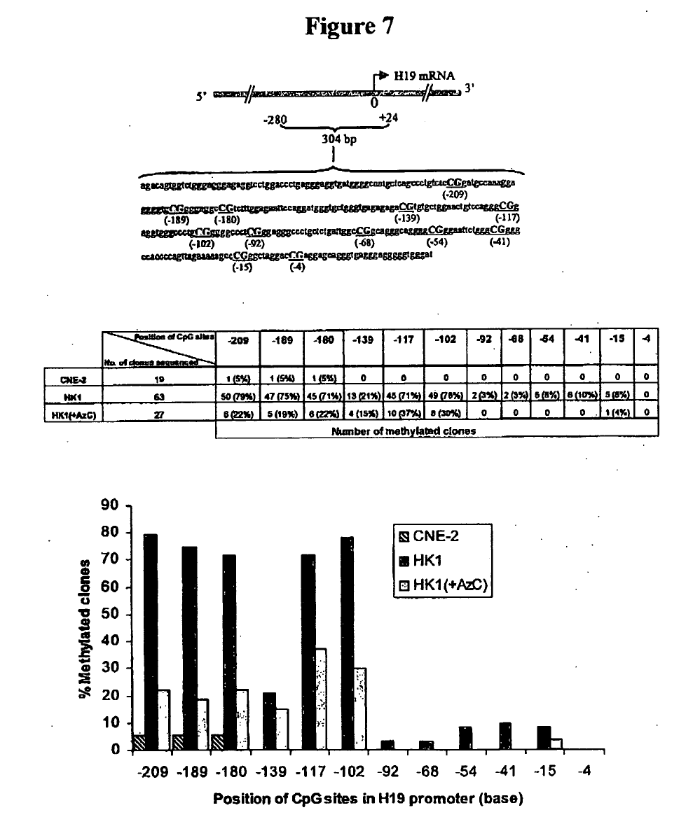

FIG. 7

The extent of CpG methylation in the 304-bp region within H19 promoter was examined using bisulphite sequencing. The methylation profile of the twelve CpG sites of this region were analysed in undifferentiated NPC cells (CNE-2), well-differentiated NPC cells (HK1) and HK1 cells treated with 5′-aza-2′-deoxycytidine (AzC), an inhibitor of methylation. The occurrence of methylation at each CpG site is expressed as a percentage of the number of clones sequenced. The number of sequenced clones derived from CNE-2, HK1 and AzC-treated HK1 cells were 19, 63 and 27 respectively.

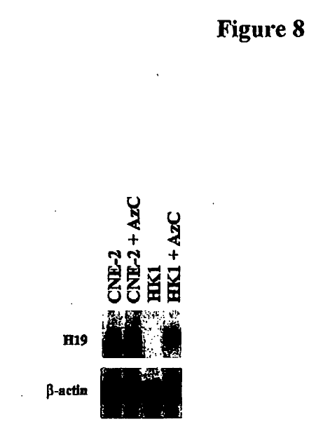

FIG. 8

Northern blot analysis to examine H19 gene expression in well-differentiated NPC (HK1) and undifferentiated NPC (CNE-2) cells, cultured for 7 days in the presence or absence of 5′-aza-2′-deoxycytidine (AzC). Blot was probed for H19 and β-actin as described in ‘Materials and Methods’.

### MATERIALS AND METHODS

Cell Lines and Tissue Culture

The human NPC cell lines CNE-2, and HK1 had been described previously (Sizhong et al., 1983; Huang et al., 1983). The CNE-2 cells were obtained from Professor H. M. Wang (Cancer Institute, Sun Yat-sen University of Medical Sciences, Guangzhou, People's Republic of China), while the cell line HK1 was obtained from Professor D. P. Huang (The Chinese University of Hong Kong). CNE-2 cells are derived from undifferentiated nasopharyngeal carcinoma (Sizhong et al., 1983), while HK1 was derived from patient with the well-differentiated squamous carcinoma of the nasopharynx (Huang et al., 1983).

Most of the tumor cell lines employed in the present study were obtained from the American Tissue Type Collection (ATCC) unless otherwise stated. These human cell lines include A498 (kidney carcinoma), A549 (lung carcinoma), DAKIKI (EBV-transformed lymphoblast), Fadu (pharyngeal carcinoma), HeLa (cervical adenocarcinoma), HepG2 (heptocellular carcinoma), MCF-7 (breast adenocarcinoma), HT-3 (cervical carcinoma), K562 (myeloid leukaemia), Detroit-562 (pharynx carcinoma), Raji (Burkitt lymphoma), WT-18 (EBV-transformed B-lymphocyte), FHS-738Lu (normal lung), MRC-5 (diploid lung). Additional cell lines employed include the SW480 (colon adenocarcinoma), PA-1 (ovarian teratocarcinoma), HeCat (epithelial), BT-20 (breast carcinoma) and Hs67 (normal thymus). All these cell lines were propagated in RPMI medium (Gibco BRL, Life Technologies, Grand Island, N.Y.) supplemented with 10% FCS (Hyclone, Logan, Utah), 0.1 mM non-essential amino acids, 4 mM L-glutamine, and 1 mM sodium pyruvate.

Tissue Speciments

Human NPC tumor biopsies were obtained prior to treatment from patients with informed consents at the Department of ENT of the Singapore General Hospital. Biopsies were obtained from patients under topical anaesthesia using 4% cocaine solution applied with a cotton swab applicator. A total of three bites of tumour tissues were taken using Hilyard forceps under direct vision with a fibre-optic naso-endoscope. The first two bites were sent for histological examination and the third biopsy obtained was taken for the present study. Tumour biopsies taken from patients were immediately snap-frozen and stored in liquid nitrogen until being studied. Histo-pathological diagnosis was confirmed in paraffin sections.

cDNA Microarrays

The inventors have selected over 1000 IMAGE human cDNA clones (Incyte Genomics Inc., Palo Alto, Calif.), representing approximately 941 distinct Unigene clusters (i.e. unique genes), for their spotted microarray studies. These 1000 clones form part of a pool of 18,000 clones established as a core facility for cDNA microarray analyses at the National Cancer Centre, Singapore. The full listing of these clones will be made available on request. These 1000 clones were streaked out and individual colonies grown overnight. Of these, 713 clones were correctly identified and verified by PCR amplification using gene-specific primer pairs. Each of the inserts was amplified from an overnight bacterial culture, using a final dilution of 1:1000 in a 100 μl PCR reaction. The PCR products were concentrated, resuspended in 20 μl of 3×SSC and then employed for printing on poly-L-lysine (Sigma Diagnostics, St. Louis, Mo.)-treated glass microscope slides (Fisher) using a robotic GMS 417 microarrayer (Genetic Microsystems Inc, Woburn, Mass.) fitted with four printing ring-pins (TeleChem International Inc, Sunnyvale, Calif.). Housekeeping genes including GAPDH, β-actin, β-2-microglobulin, cyclophilin and ubiquitin were similarly spotted as internal controls for the normalization of hybridization signals during data analysis. Following printing, the slides were inverted over a boiling water-bath (reagent grade water) for 2-3 seconds to rehydrate the array, snap-dried for 5 seconds on a 100° C. heating block for 4 seconds and cross-linked with 550 mJ ultraviolet irradiation using a Stratalinker (Strategene, La Jolla, Calif.). The slides were then placed in 0.2% SDS (10 minutes, with magnetic stirrer), followed by 5 washes in clean water (2 L) before transferring to boiling-hot water (10 minutes), blotted to remove excess liquid, desiccated for 5 minutes in 95% ethanol and air-dried for 5 minutes in an 80° C. oven.

cDNA Microarray Hybridization

The protocol accompanying the 3DNA Expression Array Detection Kit (Genisphere Inc., Montvale, N.J.) for the synthesis of hybridization probes was used, with modifications. cDNA was synthesised by reverse transcription using 10 μg of total RNA extracted from human NPC cells or from 10 μg of reference RNA (pooled from 10 cell lines) with oligo(dT) primers incorporating either the capture sequence for the 3DNA Cy5 ‘labelling’ reagent (5′- CCTGTTG CTCTATTTCCCGTGCCGCTCCGGT-(dT)ₙ-3′) or the 3DNA Cy3 ‘labelling’ reagent (5′GGCCGACTCACTGCGCGTCTTCTGTCCCGCC-(dT)ₙ-3′), respectively. The 10 cell lines from which the pooled reference RNA was generated were A498, A549, DAKIKI, CNE-2, Fadu, HeLa, HepG2, MCF-7, HT-3, and K562. cDNAs generated from each of the test RNA samples (CNE-2 or HK1) as well as the reference RNA were competitively hybridized to the microarray using a hybridization volume of 20 μl under a glass coverslip and in a dark humidified chamber (TeleChem International Inc, Sunnyvale, Calif.) overnight at 42° C.

Post-hybridisation slide washes involve a series of washes, starting with 2×SSC/0.1% SDS (2 washes, 5 minutes each), followed by 0.2×SSC/0.1% SDS (2 washes, 5 minutes each), and finally with 0.1×SSC (2 washes, 5 minutes each). The cDNA, which incorporates a fluorescent dye capture sequence, is labelled with Cy5 or Cy3 only after the cDNA has hybridised to the microarray and the excess unbound cDNA washed off.

Quantitation of Arrays and Cluster Analysis

Hybridized arrays were scanned with a GMS 418 laser scanner (Genetic Microsystems Inc, Woburn, Mass.). Images for Cy5 and Cy3 were acquired separately using different channels, superimposed and quantified with Imagene software version 3.0 (BioDiscovery Inc, Los Angeles, Calif.). Spots on the array were defined by aligning a grid of circles over each spot on the entire array image. The net signal for each spot was obtained by subtracting the background signal from the average intensity within the spot. The signal intensities obtained from both Cy5 and Cy3 channels were normalized by applying a scaling factor such that the mean Cy5:Cy3 ratio of spots across the entire array is 1.0. Log₂-transformation and centering of the median for the Cy5:Cy3 ratio were then computed. A hierarchical clustering algorithm was applied using complete linkage clustering (Gene Cluster program, http://rana.lbl.gov/; Eisen et al., 1998). The TreeView program (Eisen et al., 1998) was used to visualize the clustered data by displaying the intensity of gene expression using a spectrum of graded colors from bright red, through black, to bright green. Unfortunately, this cannot be shown in the black and white figures accompanying this specification. However, the intensities have been indicated by differently marked boxes See, for example, FIG. 1.

Northern Blot Analysis

Total cellular RNA was isolated using TRIzol Reagent (Gibco BRL, Life Technologies, Grand Island, N.Y.). Poly(A)⁺ RNA was selected by using the Fast-Track mRNA isolation kit from Invitrogen (Invitrogen Corp., San Diego, Calif.). For Northern blotting analysis, polyA⁺ RNA (5 μg) was loaded in each lane of a 1% agarose gel containing 0.7% formaldehyde and 5 mM iodoacetamide, and subjected to electrophoresis. RNA was transferred to Hybond-N⁺ nylon membrane (Amersham, Piscataway, N.J.) by capillary transfer and probed with ³²P-labelled H19 DNA (full-length cDNA clone obtained from Professor Shirley Tilghman, Princeton University, NJ). Probes were labelled by random hexa-nucleotide priming using the High Prime DNA labelling kit (Boehringer Mannheim GmbH, Mannheim, Germany) according to manufacturer's protocol. The filters employed for the human and human fetal multiple tissue Northern blot were purchased from Clontech Laboratories (Clontech Laboratories Inc., Palo Alto, Calif.). Hybridization signals were quantitated using the BioRadFX PhosphorImager (BioRad, Richmond, Calif.).

In Situ Hybridization

Frozen biopsy NPC tissues were sectioned to 10 μm in a cryostat. Cell-lines (CNE-2, HK1 and HT-3) were grown to half confluence in chambers mounted on glass slides (Falcon CultureSlide, Becton Dickinson and Co., NJ). Hybridizations were performed with non-radioactive sense and anti-sense H19 probe, which was labelled by the incorporation of digoxigenin (DIG)-labeled dUTP (DIG RNA Labelling Kit, Hoffmann-La Roche, Basel, Switzerland), according to manufacturer's instructions. The hybridized digoxigenin-labeled probes were detected with a peroxidase-conjugated anti-DIG antibody and subsequent enzyme-catalyzed color reaction with 5-bromo-4-chloro-3-indolyl phosphate and nitro blue tetrazolium salt (Boehringer Mannheim GmbH, Mannheim, Germany). Sections were counter-stained with haematoxylin (BDH Laboratory Supplies, Dorset, England). Slides were viewed with the Olympus Bx51 microscope (Olympus Optical Co. Ltd., Tokyo, Japan).

Treatment with 5′-aza-2′-deoxycytidine

Seven cell lines (CNE-2, NK1, HeLa, Hep-G2, HT-3, NIH:OVCAR-3 and SW480) were cultured separately for 7 days in RPMI (containing 10; fetal bovine serum) in the presence or absence of 12.5 μM 5′-aza-2′-deoxycytidine (sigma Diagnostics, St. Louis, Mo.). Total RNA from these cell lines was extracted using TRIzol Reagent (Gibco BRL, Life Technologies, Grand Island, N.Y.), according to manufacturer's instruction. Twenty μg of total RNA was used for Northern blot analysis.

Bisulphite Sequencing of the B19 Promoter Region

Genomic DNA (2 μg) was digested with RsaI at 37° C. for 16 h and denatured by adding freshly prepared NaOH to a final concentration of 0.3M at 42° C. for 30 min. The bisulphite reaction was carried out on the denatured DNA by adding urea/bisulphite solution and hydroquinone to final concentrations of 5.36M, 3.44M and 0.5 mM respectively. The reaction involves 20 cycles of 55° C. (15 minutes) followed by denaturation at 95° C. (30 seconds). The bisulphite-treated DNA (5 μl) was amplified by PCR in a 20 μl reaction with 0.5 units of AmpliTaq DNA polymerase (Perkin-Elmer Corp., Norwalk, Conn.) and using primers (10 μM) that will amplify a 306-bp region in the H19 promoter: 5′-AGATAGTGG TTTGGGAGGGAGAGGTTTTGGAT-3′ and 5′-ATCCCACCCCCTCCCTCACCCTACT CCTCA-3′. The reaction was subjected to 94° C. (3 minutes), then 35 cycles (of 94° C. for 30 seconds, 58° C. for 1 minute, 72° C. for 30 seconds), and ending with 72° C. (6 minutes). The bisulphite-treated DNA was then cloned and sequenced as described (Tremblay et al., 1997). DNA sequencing was carried out using a CEQ 2000 capillary sequencer (Beckman Coulter Inc., Fullerton, Calif.).

Results

The Undifferentiated Human NPC Cell Line CNE-2 and the Well-Differentiated Human HK1 NPC Tumour Cells Demonstrated Unique Gene Expression Profile

To identify human NPC-specific genes, the inventors have initiated a program at the National Cancer Centre, Singapore, to employ a library comprising 18,000 cDNA clones for the screening of human nasopharyngeal carcinoma clinical biopsies to link these expression profiles in the context of clinical information. Based on their preliminary data, they have chosen approximately 1000 genes for their present study.

Gene expression profiles were established using RNA extracted from the undifferentiated human NPC cell line CNE-2 and the well-differentiated NPC cell line HK1 and hybridized to spotted microarrays. CNE-2 and HK1 cells exhibited distinct gene expression profiles (FIG. 1, Table 1). Six genes out of the approximately 1000 genes studied were found to be consistently up-regulated in the HK1 cells in comparison to the CNE-2 cells (Table 1). These include the genes that encode metallothionein-I, human melanoma-associated antigen B3, and monocyte chemotactic protein-3 (MCP-3) (FIG. 1A, Table 1). In comparison, there are fifteen genes that were found consistently to be more highly expressed in the RNA of the undifferentiated CNE-2 cells than that of the well-differentiated HK1 cells (Table 1). Some of these genes include the H19 imprinted gene, the cyclin-dependent kinase inhibitor 1C (CDKN1C or p57KIP2) gene, genes that encode protein-tyrosine kinase Flt4, Tat-interacting protein, and cyclin D3 (FIG. 1B and C, Table 1).

H19 Gene is Highly Expressed in Undifferentiated Human NPC Cells

The specific up-regulation of the imprinted H19 gene in the undifferentiated CNE-2 NPC cells is most interesting. To examine whether the expression of H19 is unique to human NPC cells, the inventors performed Northern blot analysis to compare to expression of H19 in eighteen different human tumour cell lines of diverse origins. These include tumour cell lines that were derived from human Burkitt lymphoma, pharyngeal carcinoma, cervical carcinoma, lung carcinoma, colorectal carcinoma, ovarian teratocarcinoma, hepatocellular carcinoma, kidney carcinoma, breast carcinoma, EBV-transformed normal B lymphocytes, fibroblast, epithelium and the thymus (FIG. 2). Positive hybridization with the H19 probe could only be detected for the CNE-2 cells (FIG. 2). The other seventeen cell lines tested under these conditions did not have detectable H19 gene expression.

The specific expression of H19 in the human undifferentiated CNE-2 NPC cell line was also confirmed by in situ hybridization studies (FIG. 3). Although the expression of β-actin could be detected in the CNE-2, HK1, and HT-3 (cervical carcinoma) cells tested, the expression of H19 could only be specifically detected in CNE-2 cells (FIG. 3). The H19 mRNA expressing cells were identified by the grey-brown color staining following binding to the non-radioactive, digoxigenin-labelled anti-sense H19 RNA probe (FIG. 3).

To address the relevance of the expression of H19 in the undifferentiated CNE-2 cells, the expression of H19 in undifferentiated human primary NPC tissues by in situ hybridization studies was performed. In situ hybridization studies revealed that H19 also expressed strongly in undifferentiated human NPC biopsies (FIG. 4) and not in the epithelium of chronic inflammatory tissue biopsies that were negative for malignancy but were taken similar conditions from the nasopharyngeal region (FIG. 4). A total of seven undifferentiated human primary NPC biopsies and three non-NPC biopsies were studied by in situ hybridization and representative results were shown in FIG. 4.

Furthermore, it was also determined, by Northern blot analysis, that H19 is expressed in human placenta tissues (FIG. 5A). H19 could not be detected in RNA derived from most of the adult tissues tested. These included tissues of the heart, brain, lung, liver, skeletal muscle, kidney, pancreas, spleen, thymus, prostate, testis, ovary, small intestine, colon and leukocyte (FIG. 5A). The expression of H19 could also be detected in the RNA of fetal liver but not tissues of the fetal heart, fetal brain, fetal lung nor the fetal kidney (FIG. 5B).

H19 is a paternally imprinted gene and is located in close proximity to the maternally imprinted IGF-2 gene on chromosome 11p15.5 (Feinberg, 1999). To determine if there is a relationship between the expression of these two genes in the undifferentiated CNE-2 cells and the well-differentiated HK1 cells, Northern blot analyses were performed. In contrast to H19 that is only expressed in CNE-2 cells, IGF-2 is expressed in both CNE-2 and HK1 cells (FIG. 6).

The CpG Dinucleotides in the Promoter Region of the Well Differentiated HK1 NPC Cells are Hypermethylated

When the DNA sequence of the H19 was examined, one noticeable feature is the presence of many CpG dinucleotides in the promoter region of the H19 gene (FIG. 7). The hypo- and hyper-methylation of CpG dinucleotides have been demonstrated to be an important epigenetic event in the regulation of gene transcription. To investigate whether methylation plays a role in the regulation of the H19 gene in undifferentiated and well-differentiated NPC cells, the inventors compared the promoter methylation status of H19 gene in CNE-2 and HK1 cells. Genomic DNA were purified from CNE-2 cells and HK1 cells and the methylation status of the H19 promoter was assessed following PCR amplification of the H19 promoter region and bisulfite sequencing. Sodium bisulphite induces specifically the hydrolytic deamination of cytosine residues and not 5-methylcytosine residues. Therefore, it is expected that when the PCR-amplified DNA were sequenced following bisulphite treatment, the cytosines detected in the final sequencing reactions will represent those cytosine residues that were methylated in the native DNA sample. In comparison, all cytosine residues that were not methylated in the original DNA sample will subsequently be converted to thymine following the bisulphite treatment. A total of twelve CpG dinucleotides spanning 304bp of the H19 promoter region were studied (FIG. 7). Most of these CpG dinucleotides were not methylated in the DNA purified from the CNE-2 cells of which H19 is strongly expressed (FIG. 7). In comparison, most of these CpG dinucleotides were methylated in the genomic DNA purified from the HK1 cells of which H19 is not expressed (FIG. 7). The CpG dinucleotides at positions -209, -189, -180, -117, and -102 appear to be methylation “hot-spots” and accounted for greater than 70% of the clones sequenced (FIG. 7).

Hypomethylation of the CpG Dinucleotides within the H19 Promoter Region Correlated with the Restoration of H19 Gene Expression in the Well-differentiated HK1 NPC Cells

To determine whether the expression of the H19 gene could be induced by hypomethylation, the inventors have treated the HK1 cells with the demethylating agent 5′-aza-2′-deoxycytidine. When the RNA extracted from HK1 cells following treatment with the demethylating agent 5′-aza-2′-deoxycytidine were analyzed by Northern blot hybridization with the H19 probe, abundant amount of the H19 transcript could be detected in the RNA of the treated HK1 cells (FIG. 8).

To further address the relevance of promoter hypomethylation and the expression of the H19 gene in the HK1 cells, genomic DNA were purified from the HK1 cells following treatment with the demethylating agent 5′-aza-2′-deoxycytidine and employed for bisulfite sequencing as described above. In contrast to the CpG dinucleotides that are mostly methylated in the DNA purified from the wild type HK1 cells, the CpG dinucleotides within the H19 promoter region of the DNA purified from the 5′-aza-2′-deoxycytidine-treated HK1 cells are much less methylated (FIG. 7). These findings suggest that hypomethylation of the H19 promoter region is correlated with the expression of H19 gene in human NPC cells.

Discussion

Human NPC are classified into Types I, II, and III according to their degrees of differentiation and keratinization (Marks et al., 1998). Type I is the squamous cell NPC carcinomas that are highly differentiated and relatively less radioresponsive. Type III undifferentiated NPC carcinomas, on the other hand, are more radioresponsive (Neel 1985; larks et al., 1998). The molecular mechanism for tumor promotion and progression in human NPC is, at best, partially understood and there is no study on the relationship of the differentiation status of NPC cells and carcinogenesis. Genetic alterations have been implicated as one of many mechanisms likely to contribute towards the development of NPC. Most of these genetic alterations will be reflected by a subsequent change in the respective gene products. In Singapore, it has been suggested that more than 90% of clinically detected NPC cases are poorly differentiated. In this study, the inventors have therefore employed cDNA microarrays to identify genes whose expression differs in well-differentiated and undifferentiated NPC carcinoma cells. These genes will undoubtedly be important for elucidating human NPC carcinogenesis. From their cDNA microarray analyses, fifteen genes were demonstrated to be differentially upregulated in the undifferentiated CNE-2 NPC cells, while six genes were specifically upregulated in the well-differentiated HK1 cells (FIG. 1 and Table 1).

One of the genes that is consistently upregulated in the well-differentiated HK1 cells is metallothionein I (FIG. 1 and Table 1). Metallothionein I encodes a metal-binding protein that functions in cell growth, repair and differentiation, and has been implicated to be a potential marker for tumour differentiation or cell proliferation (Hengstler et al., 2001). Furthermore, metallothionein I also plays a protective role against DNA damage and apoptosis induced by oxidative or external stress, and has postulated to contribute towards radiation resistance in tumour cells (Jayasurya et al., 2000). Other genes that were also differentially up-regulated in HK1 cells include those encoding the monocyte chemotactic protein-3 (MCP-3), CPR2, CDK inhibitor 2A and IGFBP-3 (FIG. 1 and Table 1).

MCP-3, a C-C chemokine that interacts with chemokine receptors CCR1, CCR2, and CCR3, and is a chemo-attractant for monocytes, T cells, NK cells, eosinophils, and dendritic cells (Fioretti et al., 1998). It has been suggested that the characteristic leukocyte infiltration seen in NPC tumour lesions might be induced by C-C chemokines secreted by the infiltrating cells (Tang et al., 2001). However, the up-regulation of MCP-3 expression in the HK1 NPC cells suggested that the NPC tumor cells themselves could also contribute actively in recruiting lymphocytes to the tumour site.

Fifteen genes were found to be consistently differentially expressed at higher levels in the undifferentiated CNE-2 cells in comparison to the well-differentiated HK1 NPC cells (FIG. 1 and Table 1). One of these genes encodes protein-tyrosine kinase Flt4, a receptor-type tyrosine kinase, with which angiogenic vascular endothelial growth factor-C (VEGF-C) interacts (Lee et al., 1996). Interestingly, the enhanced expression of Flt4 in undifferentiated NPC cells concurs well with the observation that Flt4 is expressed in nondifferentiated teratocarcinoma cells but not expressed in differentiated teratocarcinoma cells (Pajusola et al., 1992). Another gene that is up-regulated in CNE-2 cells is the gene that encodes the Tat-interacting protein 30 kDa (TIP30). TIP30 is identical to CC3 that function as a suppressor of metastasis and inhibits the metastasis of human small cell lung carcinoma by promoting tumour cells to undergo apoptosis (Shtivelman 1997). This is mediated by the induction of a number of apoptosis-related genes such as Bad and Siva, and the metastasis suppressor, NM23-H2 by TIP30/CC3 (Xiao et al., 2000).

Interestingly, it was demonstrated that the H19 gene and the gene encoding CDKN1C were differentially up-regulated in the undifferentiated CNE-2 NPC cells (FIG. 1 and Table 1). Both H19 and CDKN1C genes are located at chromosome 11p15 (Feinberg, 1999) and both are reported to be imprinted genes. Genomic imprinting is a parental origin-specific chromosomal modification that causes differential expression of maternal and parental genes (Tilghman 1999). Although a relatively small number of genes has been reported to be imprinted, they nevertheless play important roles in development and carcinogenesis (Joyce and Schofield, 1998). Both the CDKN1C and H19 genes have been postulated to be tumor-suppressor genes (Hatada and Mukai, 1995). It has also been demonstrated that CDKN1C is a potent inhibitor of many G1 cyclin/Cdk complexes and a negative regulator of cell proliferation (Matsuoka et al., 1995; Hatada et al., 1996 & 1995).

H19 is a paternally imprinted gene with unknown function. It is located in close proximity to the maternally imprinted IGF-2 gene on chromosome 11p15.5 (Feinberg 1999). For normal human tissues, expression of H19 could be detected in the placenta and fetal liver tissues tested but not expressed in the other adult and fetal tissues (FIG. 5). This concurs well with studies in mouse, where the H19 gene is highly expressed in endoderm and mesoderm tissue of mouse embryos, but is dramatically down-regulated after birth (Brunkow and Tilghman, 1991).

At present, the function of H19 gene in carcinogenesis is unclear. However, the over-expression of the H19 gene in transgenic mice caused prenatal lethality in the late portion of the gestational period, strongly suggest, but does not prove, an important role for H19 during development and differentiation (Brunkow and Tilghman, 1991; Pfeifer et al., 1996). Consistent with these observations, it has been reported that the H19 gene is re-expressed in rat vascular smooth muscle cells after injury (Kim et al., 1994). There have also been a number of indications that genomic imprinting may be important in human disease (Paulsen et al., 2001). It has been reported that some patients with Beckwith-Wiedemann syndrome show uniparental disomy at 11p15 (Bliek et al., 2001). Several studies have further demonstrated the preferential retention of paternal alleles in embryonal tumours such as the Wilms' tumour (Moulton et al., 1994; Taniguchi et al., 1995) and embryonal rhabdomyosarcoma (Casola et al., 1997; Zhan et al., 1994) that had undergone loss of heterozygosity at tumour suppressor gene loci. These observations supported nonequivalence of the two alleles and suggesting a possible role for genomic imprinting in tumorigenesis (Zhang et al., 1993). It has also been shown that normal imprinting is relaxed, and gene expression is biallelic in a majority of Wilms' tumours that retain heterozygosity at this locus (Moulton et al., 1994; Taniguchi et al., 1995). The tumour-suppressor potential of the human H19 gene has also been demonstrated. Transfection of the H19 gene into two embryonal tumour cell lines abrogated the oncogenicity of some of the transformed cells in soft agar and their tumorigenicity in nude mice (Hao et al., 1993).

When the gene expression pattern of the H19 gene was examined and compared between well-differentiated and undifferentiated human NPC cells, it was demonstrated that H19 gene expression could only be specifically demonstrated in the undifferentaited CNE-2 human NPC cells (FIGS. 2, 4 and 6). This was also confirmed for human NPC biopsy tissues where H19 was expressed in undifferentiated NPC cells and not in the epithelium of normal nasopharyngeal (NP) tissues (FIG. 4). It is interesting to observe that the expression of the H19 gene differs for the two NPC cell lines that exhibited different degree of differentiation. More importantly, we demonstrated that the expression of H19 could be reversed by culturing the well-differentiated HK1 cells in the presence of 5′-aza-2′-deoxycytidine (FIG. 8). Furthermore, the expression of H19 correlated with the hypo-methylation of the CpG dinucleotides in the promoter region of the H19 gene (FIG. 7). This observation was clearly demonstrated through bisulfite DNA sequencing and is consistent with the concept that DNA methylation can modulate gene expression (Li et al., 1993, Feil and Khosla, 1999; Sleutels et al., 2000).

### REFERENCES

Bliek J, Maas Saskia, Ruijter J M, Hennekam R C M, Alders M, Westerveld A and Mannens M A M (2001) Increased tumour risk for BWS patients correlates with aberrant H19 and not KCNQ1T1 methylation: occurance of KCNQ10T1 methylation: occurance of KCNQ10T1 hypomethylation in familial cases of BWS. Hum Mol Genet 10: 467-476
Brunkow M E and Tilghman S M (1991) Ectopic expression of the H19 gene in mice causes prenatal lethality. Genes Dev 5: 1092-1101
Busson P, McCoy R, Sadler R, Gilligan R, Tursz T and Raab-Traub N (1992) Consistent transcription of the Epstein-Barr virus LMP2 gene in nasopharyngeal carcinoma. J Virol 66: 3257-3262
Camilynn I, Brannan E C D, Roberts S I and Tilgham S M (1990) The product of the H19 gene may function as an RNA. Mol & Cellul Biol 10: 28-96
Casola S. Pedone, P V, Cavazzana A O, Basso G, Luksch R, d'Amore E S, Carli M, Bruni C B, Riccio A (1997) Expression and parental imprinting of the H19 gene in human rhabdomyosarcoma. Oncogene 14: 1503-1510
Chen F F, Yan J J, Lai W W, Jin Y T and Su I J (1998) Epstein-Barr virus-associated nonsmall cell lung carcinoma: undifferentiated “lymphoepithelioma-like” carcinoma as a distinct entity with better prognosis. Cancer 82: 2334-2342
Chia K S, Seow A, Lee H P and Shanmugaratnam K (2000) Cancer incidence in Singapore 1993-1997. Singapore Cancer Registry Report 5
Chien G, Yuen P W, Kwong D and Kwong Y L (2001) Comparative genomic hybridization analysis of nasopharygeal carcinoma: consistent patterns of genetic aberrations and clinicopathological correlations. Cancer Genet. Cytogenet 126. 63-67
Eisen M B, Spellman P T, Brown P O and Botstein D (1998) Cluster analysis and display of genome-wide expression patterns. Proc Natl Acad Sci USA 95: 14863-14868
Fang Y, Guan X, Guo Y, Sham J, Deng M, Liang Q, Li H, Zhang H, Zhou H and Trent J (2001) Analysis of genetic alterations in primary nasopharyngeal carcinoma by comparative genomic hybridization. Genes Chromosomes Cancer 30: 254-260
Farrow D C, Vaughan T L, Berwick M, Lynch C F, Swanson G M and Lyon J L (1998) Diet and nasopharyngeal cancer in a low-risk population. Int J Cancer 78: 675-679
Feil R and Khosla S (1999) Genomic imprinting in mammals: an interplay between chromatin and DNA methylation? Trends Genet 15: 431-435
Feinberg A P (1999) Imprinting of a genomic domain of 11p15 and loss of imprinting in cancer: an introduction. Cancer Res 59: 1743-1746
Fioretti F, Fradelizi D, Stoppacciaro A, Ramponi S, Ruco L, Minty A, Sozzani S, Garlanda C, Vecchi A and Mantovani A (1998) Reduced tumorigenicity and augmented leukocyte infiltration after monocyte chemotactic protein-3 (MCP-3) gene transfer: perivascular accumulation of dendritic cells in peritumoral tissue and neutrophil recruitment within the tumor. J Immunol 161: 342-346
Gray J W and Collins C (2000) Genome changes and gene expression in human solid tumours. Carcinogenesis 21: 443-452
Hao Y, Crenshaw T, Moulton T, Newcomb E and Tycko B (1993) Tumour-suppressor activity of H19 RNA. Nature 366: 764-767
Hatada I, Inazawa J, Abe T, Nakayama M, Kaneko Y, Jinno Y, Niikawa N, Ohashi H, Fukushima Y, Iida K, Yutani C, Takahashi S, Chiba Y, Ohishi S and Mukai, T (1996) Genomic imprinting of human p57KIP2 and its reduced expression in Wilms' tumors. Hum Mol. Genet 5: 783-708
Hatada I and Mukai T (1995) Genomic imprinting of p57KIP2, a cyclin-dependent kinase inhibitor, in mouse. Nat Genet 11: 204-206
Hengstler J G, Pilch H, Schmidt M, Dahlenburg H, Sagemuller J, Schiffer I, Oesch F, Knapstein P G, Kaina B and Tanner B (2001) Metallothionein expression in ovarian cancer in relation to histopathological parameters and molecular markers of prognosis. Int J Cancer 95: 121-127
Huang D P, Ho J H, Poon Y F, Chew E C, Saw D, Lui M, Li C L, Mak L S, Lai S H and Lau W H (1980) Establishment of a cell line (NPC/HK1) from a differentiated squamous carcinoma of the nasopharynx. Int J Cancer 26: 127-132
Huang Y T, Sheen T S, Chen C L, Lu J, Chang Y, Chen J Y and Tsai C H (1999) Profile of cytokine expression in nasopharyngeal carcinomas: a distinct expression of interleukin 1 in tumor and CD4+ T cells. Cancer Res 59: 1599-1605
Jayasurya A, Bay B H, Yap W M and Tan N G (2000) Correlation of metallothionein expression with apoptosis in nasopharyngeal carcinoma. Br J Cancer 82: 1198-1203
Joyce J A and Schofield P N (1998) Genomic imprinting and cancer. Mol Pathol 51: 185-190
Kim D K, Zhang L, Dzau V J and Pratt R E (1994) H19, a developmentally regulated gene, is reexpressed in rat vascular smooth muscle cells after injury. J Clin Invest 93: 355-360
Lee A W M, Poon Y F, and Foo W (1992) Retrospective analysis of 5037 patients with nasopharyngeal carcinoma treated during 1976-1985. Overall survival and patterns of failure. Int J Radiat Oncol Biol 23: 261-270
Lee J, Gray A, Yuan J, Luoh S-M, Avraham H and Wood W I (1996) Vascular endothelial growth factor-related protein: a ligand and specific activator of the tyrosine kinase receptor Flt4. Proc Natl Acad Sci 92: 1988-1992
Li E, Beard C and Jaenisch R (1993) Role for DNA methylation in genomic imprinting. Nature 366: 362-365
Lo K W, Kwong J, Hui A B, Chan S Y, To K F, Chan A S, Chow L S, Teo P M, Johnson P J and Huang D P (2001) High frequency of promoter hypermethylation of RASSFlA in nasopharyngeal carcinoma. Cancer Res 61: 3877-3881
Marks J E, Phillips J L, and Menck H R (1998) The National Cancer Data Base report on the relationship of race and national origin to the histology of nasopharyngeal carcinoma. Cancer 83: 582-588
Matsuoka S, Edwards M C, Bai C, Parker S, Zhang P, Baldini A, Harper J W and Elledge S J (1995) p57KIP2, a structurally distinct member of the p21CIP1 Cdk inhibitor family, is a candidate tumor suppressor gene. Genes Dev 9: 650-662
Moulton T, Crenshaw T, Hao Y, Moosikasuwan J, Lin N, Dembitzer F, Hensle T, Weiss L, McMorrow L, Loew T, et al. (1994) Epigenetic lesions at the H19 locus in Wilms' tumour patients. Nat Genet 7: 440-447
Mutirangura A, Pornthanakasem W, Theamboonlers A, Sriuranpong V, Lertsanguansinch P V S, Voravud N, Supiyaphun P, and Poorvorawan Y (1998) Epstein-Barr viral DNA in serum of patients with nasopharyngeal carcinoma. Clin Cancer Res 4: 665-669
Neel H B 3^{rd} (1985) Nasopharyngeal carcinoma. Clinical presentation, diagnosis, treatment, and prognosis. Otolaryngol Clin North Am 18: 479-490
Neel H B 3^{rd} (1986) A prospective evaluation of patients with nasopharyngeal carcinoma: an overview. J Otolaryngol 15:137-144
Pajusola K, Aprelikova 0, Korhonen J, Kaipainen A, Pertovaara L, Alitalo R and Alitalo K (1992) FLT4 receptor tyrosine kinase contains seven immunoglobulin-like loops and is expressed in multiple human tissues and cell lines. Cancer Res 52: 5738-5743
Paulsen M and Ferguson-Smith A C (2001) DNA methylation in genomic imprinting, development, and disease. J Pathol 195: 97-110
Pfeifer K, Philip A L and Tilghman S M (1996) The structural H19 gene is required for transgene imprinting. Proc Natl Acad Sci 93: 13876-13883
Shtivelman E (1997) A link between metastasis and resistance to apoptosis of variant small cell lung carcinoma. Oncogene 14: 2167-2173
Sigel G, Schillinger M, Henninger K and Bauer G (1994) IgA directed against early antigen of Epstein-Barr virus is no specific marker for the diagnosis of nasopharyngeal carcinoma. J Med Virol 43: 222-227
Sizhong Z, Xiukung G and Yi Z (1983) Cytogenetic studies on an epithelial cell line derived from poorly differentiated nasopharyngeal carcinoma. Int J Cancer 31: 587-590
Sleutels F, Barlow D P and Lyle R (2000) The uniqueness of the imprinting mechanism. Curr Opin Genet Dev 10: 229-233
Tang K F, Tan S Y, Chan S H, Chong S M, Loh K S, Tan L K and Hu H (2001) A distinct expression of CC chemokines by macrophages in nasopharyngeal carcinoma: implication for the intense tumor infiltration by T lymphocytes and macrophages. Hum Pathol 32: 42-49
Taniguchi T, Sullivan M L, Ogawa O and Reeve A E (1995) Epigenetic changes encompassing the IGF2/H19 locus associated with relaxation of IGF2 imprinting and silencing of H19 in Wilms tumor. Proc Natl Acad Sci USA 92: 2159-2163
Thorvaldsen J L, Duran K L and Bartolomei M S (1998) Deletion of the H19 differentially methylated domain results in loss of imprinted expression of H19 and Igf2. Genes Dev 12: 3693-3702
Tilghman S M (1999) The sins of the fathers and mothers: genomic imprinting in mammalian development. Cell 96:185-193
Tremblay K D, Duran K L and Bartolomei M S (1997) A 5′ 2-kilobase-pair region of the imprinted mouse H19 gene exhibits exclusive paternal methylation throughout development. Mol Cell Biol 17: 4322-4329
Williams H K (2000) Molecular pathogenesis of oral squamous carcinoma. Mol Pathol 53: 165-172
Xiao H, Palhan V, Yang Y and Roeder R G (2000) TIP30 has an intrinsic kinase activity required for up-regulation of a subset of apoptotic genes. EMBO J 19: 956-963
Zhan S, Shapiro D N and Helman L J (1994) Activation of an imprinted allele of the insulin-like growth factor II gene implicated in rhabdomyosarcoma. J Clin Invest 94: 445-448
Zhang Y, Shields T, Crenshaw T, Hao Y, Moulton T and Tycko B (1993) Imprinting of human H19: Allele-specific CpG methylatin, loss of the active allele in Wilms tumor, and potential for somatic allele switching. Ann J Hum Genet 53: 113-124

Zong Y S, Sham J S, Ng M H, Ou X T, Guo Y Q, Zheng S A, Liang J S and Qiu H (1992) Immunoglobulin A against viral capsid antigen of Epstein-Barr virus and indirect mirror examination of the nasopharynx in the detection of asymptomatic nasopharyngeal carcinoma. Cancer 69: 3-7

TABLE 1
Summary of cDNA Microarray analyses.
Identification of genes that are differentially expressed
in well-differentiated and undifferentiated NPC cells.

| Genes expressed at higher levels in CNE-2 | Genbank | IMAGE |
|---|---|---|
| than in HK1 cells | | |
| H19 mRNA, NIH_MGC_10 Homo sapiens | BE018809 | 304917 |
| cDNA clone cyclin-dependent kinase inhibitor | AW612762 | 295700 |
| 1c; p57-KIP2; CDKN1C | | |
| Protein-tyrosine kinase Flt4 | A1598102 | 222781 |
| interferon, gamma-inducible protein 30 | AA527870 | 965434 |
| pre-B-cell leukemia transcription factor 1 | AA223573 | 650807 |
| thioredoxin-dependent peroxide reductase 1 | H69143 | 212165 |
| (NK-enhancing factor B) | | |
| Tat-interacting protein 30 kDa | A1161117 | 172100 |
| CDK2 (cell division protein kinase 2), | AW572951 | 293210 |
| 8CL2-antagonist of cell death | A1245965 | 187192 |
| bcl-7B protein | AW303330 | 281343 |
| death-associated protein | W46901 | 324439 |
| cyclin D3 | AW316802 | 282772 |
| connective tissue growth factor precursor | AI952812 | 249116 |
| Rho GDP dissociation inhibitor (GDI) beta | AA188078 | 624801 |
| cathepsin L precursor; major excreted protein | AW572137 | 275065 |
| (MEP) | | |
| Genes expressed at higher levels in HK1 than | Genbank | IMAGE |
| in CNE-2 cells | | |
| IGPBP3, Insulin-like growth factor binding | AW613832 | 296890 |
| protein 3 | | |
| cell cycle progression 2 protein (CPR2) | AW518910 | 287672 |
| metallothionein-Ie (hMT-Ie) | W73154 | 344345 |
| cyclin-dependent kinase inhibitor 2A | AI859822 | 243653 |
| (melanoma, p16, inhibits CDK4) | | |
| monocyte chemotactic protein 3 precursor | BE046143 | 312647 |
| (Human) | | |
| Human melanoma-associated antigen B3 | A1954607 | 247315 |

1. A method for determining the presence or risk of a nasopharyngeal carcinoma (NPC) in an individual, comprising the steps of
(a) obtaining expression products from a nasopharyngeal cell obtained from said individual suspected of having or at risk of having NPC;
(b) contacting said expression products with one or more binding members capable of binding to expression products or one or more genes identified in Table I; and
(c) determining the presence or risk of NPC in said patient based on the binding of the expression products from said nasopharyngeal cell to the one or more binding members.

2. A method for determining the type of nasopharyngeal carcinoma (NPC) in an individual, comprising the steps of
(a) obtaining expression products from a nasopharyngeal cell obtained from said individual suspected of having or at risk of having NPC;
(b) contacting said expression products with one or more binding members capable of binding to expression products or one or more genes identified in Table I; and
(c) determining the type of NPC in said patient based on the binding of the expression products from said nasopharyngeal cell to the one or more binding members.

3. A method according to claim 1 or claim 2 wherein the expression product is a transcribed nucleic acid sequence.

4. A method according to claim 3 wherein the transcribed nucleic acid sequence is RNA, mRNA or cDNA produced from mRNA.

5. A method according to any one of the preceding claims wherein the binding member is a nucleic acid sequence capable of specifically binding to the transcribed nucleic acid sequence.

6. A method according to claim 1 or claim 2 wherein the expression product is an expressed polypeptide.

7. A method according to claim 6 wherein the binding member is an antibody, or a substance comprising an antibody binding domain, which is capable of specifically binding said expressed polypeptide.

8. A method according to any one of the preceding claims wherein the binding member is labelled for detection purposes.

9. A method according to any one of the preceding claims wherein the one or more binding members are fixed to a solid support.

10. A method according to any one of claims 1 to 8 comprising fixing the expression products to a solid support.

11. A method of creating an expression profile characteristic of NPC, or a particular type of NPC, said method comprising the steps of
(a) obtaining expression products from a NPC cell obtained from an individual;
(b) contacting said expression products with a plurality of binding members capable of specifically binding to expression products of one or more genes identified in Table I;
(c) determining the binding of said expression products with the binding members so as to create an expression profile characteristic of the NPC cell.

12. A method of creating an expression profile characteristic of NPC, or a particular type of NPC, said method comprising the steps of
(a) obtaining expression products from a NPC cell and expression products from a normal nasopharyngeal cell;
(b) contacting said expression products of said NPC cell and said normal cell respectively with a plurality of binding members capable of specifically binding to expression products of one or more genes identified in Table I;
(c) comparing the expression profile of the NPC cell and the normal cell; and
(d) determining an expression profile characteristic of the NPC cell.

13. A method according to claim 11 or claim 12 wherein the expression product is a transcribed nucleic acid sequence.

14. A method according to claim 13 wherein the transcribed nucleic acid sequence is RNA, mRNA or cDNA produced from mRNA.

15. A method according to any one of claims 11 to 14 wherein the binding member is a nucleic acid sequence capable of specifically binding to the transcribed nucleic acid sequence.

16. A method according to claim 11 or claim 12 wherein the expression product is an expressed polypeptide.

17. A method according to claim 16 wherein the binding member is an antibody, or a substance comprising an antibody binding domain, which is capable of specifically binding said expressed polypeptide.

18. A method according to any one of claims 11 to 17 wherein the binding member is labelled for detection purposes.

19. A method according to any one of claims any one of 11 to 18 wherein the one or more binding members are fixed to a solid support.

20. A method according to any one of claims 11 to 18 further comprising fixing the expression products to a solid support.

21. A diagnostic reagent comprising a solid support on to which is fixed one or more binding members capable of specifically binding to an expression product of one or more genes identified in Table I.

22. A diagnostic reagent according to claim 21 wherein the one or more binding members include a binding member capable of specifically binding to an expression product of H19 or CDKNIC.

23. A diagnostic reagent according to claim 21 or claim 22 wherein the expression products are mRNA or the resulting protein product.

24. A kit for determining the presence or type of NPC in a biological sample, said kit comprising a diagnostic reagent according to any one of claims 21 to 23 and a detection means.

25. A kit according to claim 24 wherein the biological sample is cell extract.

26. A kit according to claim 24 or claim 25 wherein the detection means is a label that detects when a binding member has bound to an expression product.

27. Use of a demethylation agent in the preparation of a medicament for treating an individual with or at risk from NPC, said treatment being in association with a second cancer treatment.

28. Use according to claim 27 wherein the second cancer treatment is chemo or radiotherapy.

29. Use according to claim 27 or claim 28 wherein the demethylation agent is 5′aza-2′-deoxycytidine.

30. Use according to any one of claims 27 to 29 wherein the NPC is type I.

31. A method for treating an individual with or at risk from NPC comprising administering to said individual a demethylation agent in association with a second cancer treatment.

32. A method according to claim 31 wherein the second cancer treatment is chemo or radiotherapy.

33. A method according to claim 31 or claim 32 wherein the demethylation agent is 5′aza-2′-deoxycytidine.

34. A method according to any one of claims 31 to 33 wherein the NPC is type I.

35. A method of screening for substances capable of treating NPC in an individual said method comprising
(a) over-expressing in a cell one or more genes identified in Table I;
(b) contacting said cell with a test substance;
(c) determining the effect of said test substance on said cell as compared to the effect of said test substance on a comparable cell absent of the over-expression of said one or more genes; and
(d) identifying said test substance as a substance capable of treating NPC.

36. A method according to claim 35 wherein the one or more genes are overexpressed by inserting into said cell nucleic acid capable of expressing expression products characteristic of said genes.

37. A method according to claim 35 or claim 36 wherein said one or more genes are up-regulated in differentiated NPC.

38. A method according to claim 35 or claim 36 wherein said one or more genes are up-regulated in undifferentiated NPC.

39. A method according to claim 38 wherein the one or more genes include H19 and CDKNIC.

40. A method according to any one of claims 35 to 39 further comprising treating the cell over-expressing the one or more genes identified in Table I with a demethylation agent.

41. A method according to claim 35 wherein the cell over-expressing one or more genes identified in Table I is an NPC cell.

42. A method according to any one of claims 35 to 41 further comprising the step of producing a pharmaceutical composition comprising the substance identified in step (d).
